(19) United States
(12) Patent Application Publication
(10) Pub. No.: US 2009/0042300 A1
(43) Pub. Date: Feb. 12, 2009

(54) METHOD FOR ENHANCING GENE EXPRESSION IN PLANTS

(75) Inventors: Wyatt Paul, Pont du Chateau (FR); Thierry Risacher, Cambridge (GB); Baptiste Lelong, Orcet (FR)

Correspondence Address: CONNOLLY BOVE LODGE & HUTZ, LLP, P O BOX 2207, WILMINGTON, DE, 19899, US

(73) Assignee: BIOGEMMA S.A.S., Paris (FR)

(21) Appl. No.: 11/631,742

(22) PCT Filed: Jul. 1, 2005
(86) PCT No.: PCT/EP2005/053148

§ 371 Date: Oct. 13, 2008

(30) Foreign Application Priority Data
Jul. 6, 2004 (EP) 04103188.1

(51) Int. Cl. C12N 15/11

(52) U.S. Cl. 435/468; 536/24.1

(57) ABSTRACT

The invention relates to the field of gene expression in plants and describes methods and constructs using the first intron of a FAD2 gene in order to enhance gene expression.

### BACKGROUND OF THE INVENTION

Efficient transformation systems are available in plants, in particular in monocot species especially those of economic importance such as maize, wheat, barley and rice. It is now possible to contemplate the introduction of a range of traits by such technique. The most widely used techniques are the delivery of naked DNA by micro projectile bombardment or the use of Agrobacterium as a vector. In both cases the gene of interest and associated sequences are introduced into a suitable target plant tissue and become stably integrated into the plant genome. The target tissue has the potential to regenerate into a whole, fully-fertile plant.

In order for the introduced gene to be expressed, it must be driven by a promoter. Promoters can be of different types; constitutive or tissue and temporally specific. Widely use promoters include those derived from virus and bacteria; including Cauliflower Mosaic Virus 35S and octopine and nopaline synthase and RuBISCo, actin and ubiquitin from plant species.

The most widely used promoters are constitutive because they are generally used to control the selectable marker gene and can also be used to drive the gene of interest.

Although suitable promoters for monocot species are known, insufficient numbers are available for use in biotechnology applications. Transformation may require that one promoter drives the selectable marker and a separate promoter drives the gene of interest.

It is indeed undesirable to use the same promoter for both genes for several reasons. Initially the cloning and construct preparation becomes problematical. Sequence duplications can lead to gene deletions and other corruptions which would result in elimination of gene expression. Sequence duplication subsequently in planta can result in gene silencing. In a longer term consideration of breeding programmes, either gene stacking by retransformation or crossing of existing transgenic lines, the lack of choice of promoters will become increasingly limiting.

Strong promoters are preferred as the efficiency of production of useful transgenic plants is increased, in two ways. Firstly more plants are initially selected in tissue culture due to sufficient expression of the selectable marker to survive the selection conditions and secondly more plants thus selected will have a good phenotype resulting from the expression of the gene or genes of interest. In order to increase the number of suitable promoters it was discovered that the strength of a promoter can be increased by use of an intron.

Introns interrupt the coding regions of Eukaryotic genes and are removed post-transcription by the process of splicing. The expression of genes in animal cells has been known to be intron dependant since the late 1970s but the first demonstration of the effect of the inclusion of an intron in a plant species was not until 1987 (Callis et al, Genes and Development 1: 1183-1200).

The most widely used introns are the first introns of rice Actin and maize Ubiquitin, used in combination with the natural promoters. These promoters were the first strong promoters identified for monocot species. Rice actin was initially identified as a suitable candidate gene for provision of a strong constitutive promoter because actin is a fundamental and essential component of the eukaryotic cell and cytoskeleton. A rice actin gene was identified that encoded a transcript that was abundant in all rice tissues at all developmental stages (McElroy et al 1990, Plant Cell 2:163.) Further characterisation revealed that the presence of the first intron was essential for the efficient function of the promoter per se. Similarly the maize ubiquitin promoter was identified as having a high level constitutive activity dependant on the presence of its own first intron (Morris et al 1993, Plant Molecular Biology 21: 895-906).

The enhancing effect of the rice actin first intron and the maize ubiquitin first intron can also be combined with heterologous promoter sequences which alone have minimal activity in monocot species. (Vain et al, 1996, Plant Cell Reports 15: 489-494)

A limited number of plant introns have been identified that can be combined with suboptimal heterologous promoters to create novel promoters sufficiently strong to use for biotechnological applications. Early comparisons of a range of introns revealed that not all have a significantly enhancing activity in combination with a suboptimal promoter. (Vain et al, 1996)

Nevertheless, intron duplication is undesirable for same reasons that promoter duplication should be avoided.

There is therefore a continuing need to identify new suitable introns to enhance expression in plants, and especially in monocot transformation systems. These introns need to be able to increase the level of expression when combined with various promoters.

At the present time there are very few such introns identified and this ultimately limits the number of genetic transformations that can be carried out in a given monocot species.

The invention now provides a new intron that is usable for increasing expression of an heterologous gene in a plant cell, and in particular a monocot plant cell, such as wheat, maize, barley, or rice.

### SUMMARY OF THE INVENTION

The inventors have indeed surprisingly observed that presence of the first intron of the FAD2 gene, which was believed to have no effect on expression, can effectively enhance the efficiency of a promoter.

The FAD2 gene is the gene coding for the microsomal omega-6 fatty acid desaturase (FAD2/delta-12 desaturase), and its sequence is well known by the person skilled in the art, with the availability of the sequence of the chromosome 3 Arabidopsis thaliana genome.

Other FAD2 genes have been identified (for example Brassica oleracea, represented by AF181726 in GenBank, soybean with GenBank protein accession number P48628, Brassica napus, GenBank protein accession number P48627 . . . ). The identification of the first introns of FAD2 genes of other species is within the skill of the person in the art, by the differences between the cDNA and genomic sequences.

### FIGURES

FIG. 1: Schematic representation of pJIT65del

FIG. 2: Schematic representation of pWP443A

FIG. 3: Schematic representation of pWP461

FIG. 4: Schematic representation of pWP464

FIG. 5: Schematic representation of pWP480

FIG. 6: Schematic representation of pWP500

FIG. 7: Schematic representation of pWP514

FIG. 8: Transient expression comparison in wheat. Embryos bombarded with 3 promoters with and without FAD2 intron.

FIG. 9: Transient expression comparison in barley a) embryos bombarded with pWP480 b) embryos bombarded with pWP500

FIG. 10: Leaves from wheat stably transformed wheat with a) pBSV without FAD2 intron, or b) pBSV with FAD2 intron.

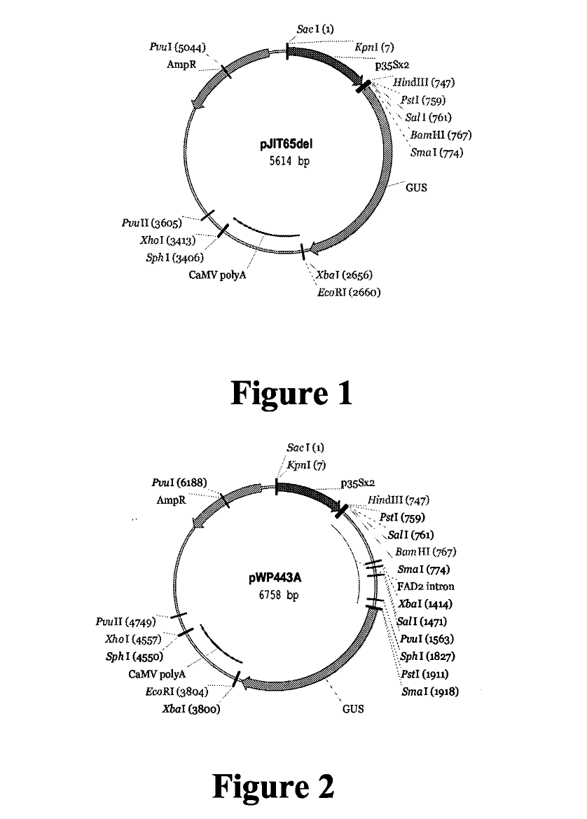

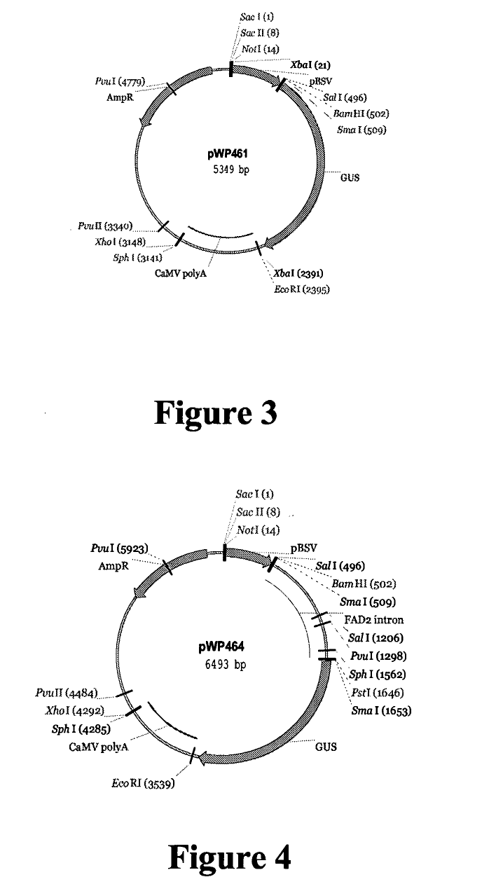

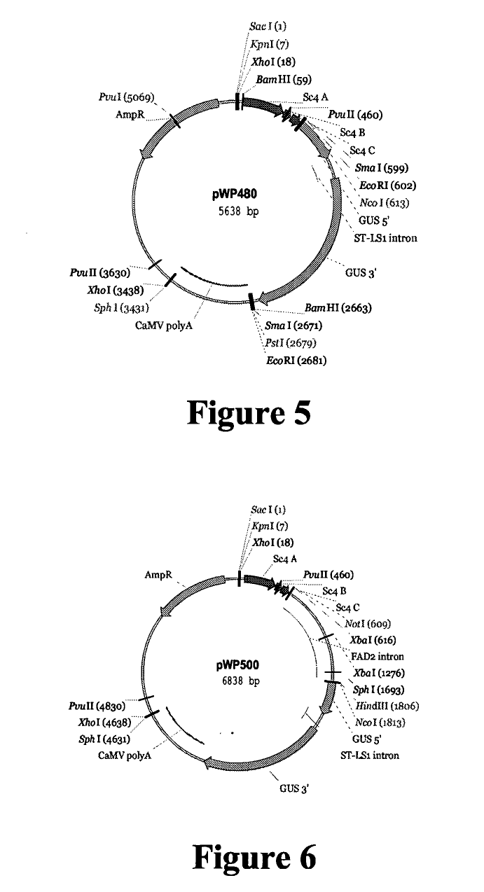

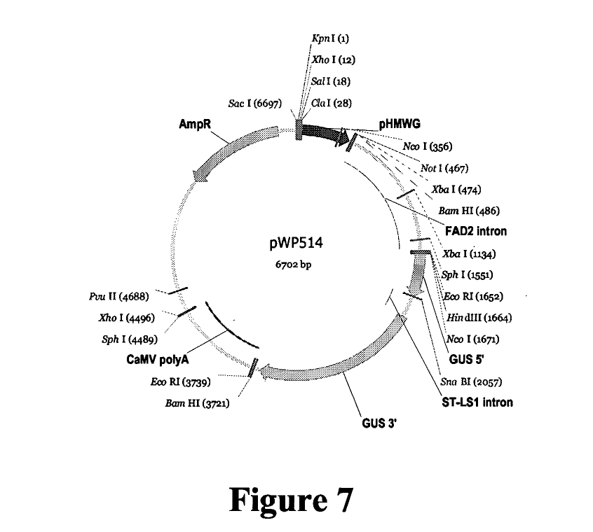

### DETAILED DESCRIPTION OF THE INVENTION

The invention thus relates to a genetic construct for the expression of a polypeptide in a plant cell, comprising:
(a) a promoter operative within said plant cell,
(b) all or part of the first intron of a FAD2 gene,
(c) a nucleotide sequence operably linked to said promoter, wherein said nucleotide sequence encodes said polypeptide,
(d) a polyadenylation site (PAA) operably linked to said nucleotide sequence.

Said polynucleotide is called “polynucleotide of interest” and is coded by a “gene of interest”.

A “promoter operative in a plant cell” is intended to mean a sequence that directs the initiation of transcription in a plant cell. It can derive from one or multiple sources, be natural or synthetic.

In the preferred embodiment, the genetic construct of the invention is such that said FAD2 gene is from Arabidopsis thaliana (GenBank AJ271841), wherein said first intron is represented by SEQ ID No 1, or as indicated in SEQ ID No 3, 5, 7 and 8. it is also envisaged that said FAD 2 intron has 80, 90 or 95% identity with SEQ ID No 1, over at least 500 or 1000 nucleotides.

Nevertheless, other FAD2 first introns from other species may also be used, such as from maize, wheat, barley, rape, canola, sunflower, potato . . . .

In the context of the present invention, “all or part of the first intron of a FAD2 gene” is to be considered as the full sequence of the first intron of a FAD 2 gene, or a fragment comprising at least 200, 500 or 1000 nucleotides of said sequence.

The promoter could be any promoter, as described above. In a preferred embodiment, said promoter is a tissue-specific promoter. In another embodiment, said promoter is a constitutive promoter. Thus it is clear that the envisaged promoter is preferably a heterologous promoter, i.e. different of the natural FAD2 promoter.

Promoter sequences of genes which are expressed naturally in plants can be of plant, bacterial or viral origin. Suitable constitutive promoters include but are not restricted to octopine synthase, nopaline synthase, mannopine synthase derived from the T-DNA of Agrobacterium tumefaciens; CaMV35S and CaMV19S from Cauliflower Mosaic Virus; rice actin, maize ubiquitin and histone promoters from plant species.

Tissue specific promoters include but are not restricted to rolC from Agrobacterium rhizogenes; RTBV from Rice Tungro Bacilliform Virus; LMW and HMW glutenin from wheat; alcohol dehydrogenase, waxy, zein from maize and, AoPR1 from Asparagus.

All these promoters (and others) are well described in the art.

Whilst the most widely used promoters are constitutive or tissue specific it is also possible to contemplate the use of inducible promoters such as PinII or AoPRT-L (WO 99/66057); more specifically light inducible including RUBISCO small subunit and chlorophyll a/b binding protein from a range of species and synthetic promoters eg EMU (US19900525866).

The genetic construct according to the invention is introduced within the plant cell on a vector. In an embodiment, said vector is maintained as an episomal vector within the plant cell. In the preferred embodiment, said vector is capable of integration within the genome of said plant cell. Integration of a genetic construct within a plant cell is performed using methods known by those skilled in the art, for example transformation by Agrobacterium, or gun bombardment.

Agrobacterium tumefaciens has been widely and efficiently used to transform numerous species dicots and including monocots such as maize, rice, barley and wheat (WO 00/63398). Alternative gene transfer and transformation methods include protoplast transformation through calcium, polyethylene glycol or electroporation mediated uptake of naked DNA. Additional methods include introduction of DNA into intact cells or regenerable tissues by microinjection, silicon carbide fibres or, most widely, microprojectile bombardment. All these methods are now well known in the art.

The invention also relates to a method for enhancing the expression of a polypeptide in a plant cell, comprising the step of introducing the genetic construct of the invention within said plant cell, for example using the methods as mentioned above. Expression is enhanced as compared to expression obtained when the FAD2 intron is not present within the construct.

Expression relates to transcription and translation of a gene so that a protein is made.

In another aspect, the invention relates to a method for increasing the expression rate of a gene, under the control of a promoter in a plant cell, said method comprising the steps of:
(a) inserting the first intron of the FAD 2 gene between said promoter and the translation start of said gene, thereby forming an intron-modified gene; and
(b) introducing said intron-modified gene into a plant cell such that said gene is expressed under the control of said intron in said plant cell, whereby the expression rate of said gene is increased as compared to an unmodified gene.

Said gene is called “gene of interest”.

In particular, said gene can be a selectable marker. These selectable markers include, but are not limited to, antibiotic resistance genes, herbicide resistance genes or visible marker genes. Other phenotypic markers are known in the art and may be used in this invention.

A number of selective agents and resistance genes are known in the art. (See, for example, Hauptmann et al., 1988; Dekeyser et al., 1988; Eichholtz et al., 1987; and Meijer et al., 1991).

Notably the selectable marker used can be the bar gene conferring resistance to bialaphos (White et al., 1990), the sulfonamide herbicide Asulam resistance gene, sul (described in WO 98/49316) encoding a type I dihydropterate synthase (DHPS), the nptII gene conferring resistance to a group of antibiotics including kanamycin, G418, paromomycin and neomycin (Bevan et al., 1983), the hph gene conferring resistance to hygromycin (Gritz et al., 1983), the EPSPS gene conferring tolerance to glyphosate (U.S. Pat. No. 5,188,642), the HPPD gene conferring resistance to isoxazoles (WO 96/38567), the gene encoding for the GUS enzyme, the green fluorescent protein (GFP), expression of which, confers a recognisible physical characteristic to transformed cells, the chloramphenicol transferase gene, expression of which, detoxifies chloramphenicol.

In another embodiment, said gene encodes a protein to impart insect resistance, more preferably genes which encode for Bacillus thuringiensis (Bt) endotoxins (inter alia, U.S. Pat. Nos. 5,460,963; 5,683,691; 5,545,565; 5,530,197; 5,317,096). The nucleic acids that are preferably embraced by the instant invention are cryI, cryII, cryIII, and cryIV genes. More preferably, the genes include: cry1A(a), cryIA(b); cryIA(c); and cryIIIA(a). Most preferably the gene is cryIA(a), cryIA(b) or cryIA(c).

Other genes of interest are
the bacterial gene dapA for increasing the level of lysine;
the gene for endotoxin Bt or for a protease inhibitor or for proteins extracted from bacteria such as Photorabiis (WO 97/17432 & WO 98/08932), for resistance to insects;
among the proteins or peptides of interest which confer novel properties of resistance to diseases, mention will be made in particular of chitinases (WO 92/01792), glucanases (WO 93/02197), oxalate oxidase (WO 94/13790) or antibacterial and/or antifungal peptides, in particular the peptides of less than 100 amino acids rich in cysteins, such as plant thionins or defensins, and more particularly lytic peptides of any origins comprising one or more disulfide bridges between the cysteins and regions comprising basic amino acids, in particular the following lytic peptides: androctonin (WO 97/30082 and WO 99/09189), drosomicin (WO 99/02717), thanatin (WO 99/24594) or heliomycin (WO 99/53053). According to a particular embodiment of the invention, the protein or peptide of interest is chosen from fungal elicitor peptides, in particular elicitins (Kamoun et al., 1993; Panabières et al., 1995).
genes involved in the biosynthetic processes which lead to a change in the quality of the products of the transgenic plant, such as the genes encoding enzymes for the biosynthesis or degradation of starch (i.e. synthases, starch-branching enzymes, etc.); genes encoding grain storage proteins (i.e. subunits of glutenins, gliadins, hordeins); genes related to the strength of the grain in wheat (i.e. puroindolines).
genes which modify the constitution of the modified plants, in particular the content and the quality of certain essential fatty acids (EP 666 918) or the content and the quality of the proteins, in particular in the leaves and/or the grains of said plants. Mention will be made, in particular, of the genes encoding proteins enriched in sulfur-containing amino acids (Korit, A. A. et al.; WO 98/20133; WO 97/41239; WO 95/31554; WO 94/20828; WO 92/14822). The function of these proteins enriched in sulfur-containing amino acids will also be to trap and store excess cystein and/or methionine, making it possible to avoid the possible problems of toxicity linked to an overproduction of these sulfur-containing amino acids by trapping them. Mention may also be made of genes encoding peptides rich in sulfur-containing amino acids, and more particularly in cysteins, said peptides also having antibacterial and/or antifungal activity. Mention will be made more particularly of plant defensins, and also lytic peptides of any origin, and more particularly the following lytic peptides: androctonin (WO 97/30082 and WO 99/09189), drosomicin (WO 99/02717), thanatin (WO 99/24594) or heliomycin (WO 99/53053).
genes for artificial male sterility (i.e. barnase, and PR-glucanase under the control of a suitable promoter) may also be used for the production of hybrid seeds.

The invention also relates to a method as mentioned above wherein said intron has a nucleotide sequence as set forth in SEQ ID No 1 or functionally equivalent duplications or modifications in length or minor sequence variations that do not significantly affect function, which is expression enhancing. It is well within the skills of a person skilled in the art to modify the FAD 2 intron sequence and test new constructs for their ability to improve expression of a gene, various protocols in the art, and in particular the ones described in the examples.

In another aspect, the invention relates to the use of all or part of the first intron of the FAD2 gene for enhancing expression of a polypeptide in a plant cell.

In another aspect, the FAD2 first intron may be used to convert a tissue specific promoter into a constitutive promoter. For example, the strong seed specific HMW glutenin promoter is converted into a strong/useful promoter active in many tissue types, when used with the FAD 2 first intron.

### EXAMPLES

All DNA modifications and digestions were performed using enzymes according to the manufacturers' instructions and following protocols described in Sambrook and Russell, 2001; Molecular Cloning, A Laboratory Manual.

### Example 1

### Cloning of FAD 2 Intron

The first intron of the FAD 2 gene was obtained by the polymerase chain reaction (PCR) from Arabidopsis thaliana genomic DNA. The following pair of primers were used:

| | | |
|---|---|---|
| GGGCCAGGTCCGTCGCTTCTCTTCC | FADfor | (SEQ ID N^{o} 9) |
| GGGTTTCTGCAGAAAACCAAAAGC | FADrev | (SEQ ID N^{o} 10) |

A standard PCR reaction was carried out, with amplification under the following conditions: 30 cycles of 97° C. for 30 seconds, 57° C. for 30 seconds and 74° C. for 1 minute 40 seconds. The reaction products were separated by agarose gel electrophoresis.

The desired 1146 bp product was excised from the gel and ligated into the SmaI restriction site of pTZ18 (standard cloning vector; Pharmacia) to create pWP430. The fidelity of the clone was confirmed by sequencing.

### Example 2

### Preparation of Gus Constructs

Several promoters, which are known in the field of plant biotechnology were identified, to be tested with and without the FAD2 intron. These included but were not limited to Cauliflower Mosaic Virus 35S (Odell et al, Nature. 1985 Feb. 28-Mar. 6; 313(6005):810-2), Banana Streak Virus promoter of ORF1 (WO 99/43836), Sc4, a member of the Plant Expression (PLEX) promoter family (EP 785999; U.S. Pat. No. 6,211,431) and HMW glutenin (HMWG) promoter. Constructs were prepared from well-characterised genetic elements and cloned into widely used plasmid vectors.

35S: pJIT65del

The 35S promoter was cloned in a pUC vector, driving the GUS gene (Jefferson et al, PNAS 83: 8447-8451, 1986), to obtain plasmid pJIT65. This plasmid was modified by digestion with XbaI and EcoRI to removing intervening BamHI and SmaI sites. This modified version was named as pJIT65del.

35S+FAD2 Intron: pWP443A

The FAD2 intron was inserted between the 35S promoter and the GUS gene, specifically into the 5′UTR, by digesting pWP430 with SmaI and ligating the intron into SmaI digested pJIT65del, to create pWP443A.

BSV: pWP461

The BSV promoter was cloned into the EcoRV site of Bluescript KS to create pWP453B. The following primers were used:

| | | |
|---|---|---|
| GATGCTCTAGATCTGCTGG | BSVFor | (SEQ ID N^{o} 11) |
| GGCCATGGCTTGCTCTGATACCAGACGGG | BSVRev | (SEQ ID N^{o} 12) |

pWP453B was digested with SstI and SalI to isolate the BSV promoter fragment which was then ligated into SstI and SalI digested JIT65del, thus replacing the 35S promoter to create pWP461.

BSV+FAD2 Intron: pWP464

To create the combination of the BSV promoter with the FAD2 intron the 35S promoter of pWP443A was replaced by the BSV promoter from pWP461. Both plasmids were digested with SstI and BamHI and fragments ligated to produce pWP464.

Sc4: pWP480

A previously prepared construct, pKH2, having the Sc4 promoter driving the GUS gene, with the inclusion of the rice actin first intron in the 5′UTR, was digested with SstII and NcoI to remove the actin intron and create pWP480.

Sc4+FAD2 Intron: pWP 500

Similarly to the creation of pWP464, to combine the Sc4 promoter with the FAD2 intron the 35S promoter of pWP443A was replaced by the Sc4 promoter from pWP480 using suitable digests.

HMWG+Intron: pWP514

A pUC clone of the HMWG promoter driving the GUS gene (Glu-1Dx5-GUS2) was obtained as described in Halford et al 1989 (Plant Science 62, 207-16). The FAD2 intron was inserted between the HMWG promoter and the GUS gene, specifically into the 5′UTR, to create pWP514.

### Example 3

### Transient Expression in Wheat

### a) Initial Assessment of Functioning of Constructs

A range of plant tissues and calli were bombarded with particles coated with one of the four promoter constructs including the FAD2 intron, according to methods known in the art. The tissues were immersed in a solution of the histochemical substrate. X-glucuronide, and a subjective assessment was made of the activity of the construct. In each case a significant number of blue spots were observed on each of the tissues tested.

| | TRANSIENT EXPRESSION |
|---|---|
| pWP443A | Strong expression on embryo, leaf and callus |
| pWP500 | Strong expression on embryo, leaf and callus |
| pWP464 | Strong expression on embryo, leaf and callus |
| pWP514 | Good expression on embryo, leaf and endosperms |

It is especially worthy of note that there was significant expression in leaf and embryo tissues following bombardment with the HMWG construct (pWP514). The HMWG promoter is naturally strong endosperm-specific promoter. The inclusion of the FAD2 intron has converted it into a strong constitutive promoter.

b) Comparison of Constitutive Promoters with and without Introns

Having established that the promoters modified by the inclusion of the FAD2 intron were functioning a quantitative comparison was made between the promoters with and without the intron.

Isolated embryos were arranged in 4×4 arrays. Thirty-two embryos were bombarded with each construct.

Co-bombardment with a second construct containing the Green Fluorescent Protein (GFP) under control of the rice actin promoter, was performed. Expression of GFP can be non-destructively tested and hence the relative efficiency of each individual bombardment can be normalized to allow comparison between individual bombardments.

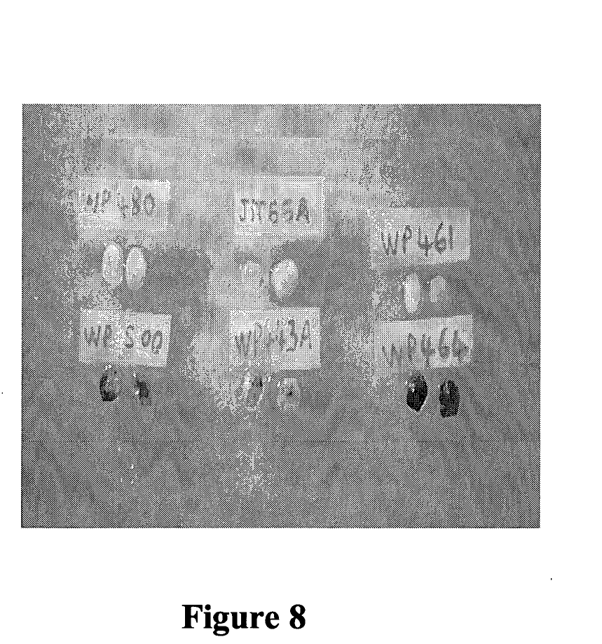

The level of expression for each construct was again visually assessed by histochemical assay for the GUS gene (FIG. 8) which enabled both a quantitative (counting the number of blue foci by 4×4 array), and a qualitative assessment (consideration of the size of blue foci).

The increase in expression due to inclusion of the FAD2 intron was 5.6 fold for the Sc4 promoter, 8 fold for the double 35S promoter and most remarkably 161 fold for the BSV promoter. Thus the expression of the weakest promoter (BSV) is increased to a level comparable with the maize ubiquitin promoter (pAAA) which is widely used because it is known to be very strong.

| | pSc4 | | | | pBSV | | | | p35S | | | | Positive Control | |
|---|---|---|---|---|---|---|---|---|---|---|---|---|---|---|
| | pWP480 | | pWP500 | | pWP461 | | pWP464 | | pJIT65del | | pWP443A | | pAAA | |
| GFP | 1648 | 698 | 1501 | 1945 | 989 | 1015 | 1504 | 1384 | 470 | 922 | 711 | 687 | 1502 | 1390 |
| GUS | 610 | 142 | 2340 | 3101 | 36 | 14 | 5067 | 4287 | 93 | 296 | 1539 | 1279 | 7200 | 7200 |
| Note on | Very small spots, | | Big spots, well | | Very small | | Very big, | | Very small | | Plenty of rather | | Very big spots, | |
| GUS spots | difficult to see | | seen but no | | spots (two big | | ‘spreading’ | | dots, | | big spots | | ‘spreading’ all | |
| | with the naked | | spreading nor | | spots on two | | spots | | sometimes not | | | | over the embryo | |
| | eye | | covering | | different | | | | seen with the | | | | difficult to score | |
| | | | | | embryos) | | | | naked eye, few | | | | | |
| | | | | | | | | | big ones | | | | | |
| Ratio | 0.37 | 0.20 | 1.55 | 1.59 | 0.03 | 0.01 | 3.36 | 3.09 | 0.19 | 0.32 | 2.16 | 1.86 | 4.79 | 5.17 |
| GUS/GFP | | | | | | | | | | | | | | |

| | | | | | | | |
|---|---|---|---|---|---|---|---|
| Average | 0.28 | 1.57 | 0.02 | 3.22 | 0.25 | 2.01 | 4.98 |

| | | | | |
|---|---|---|---|---|
| Ratio | 5.6 | 161 | 8 | / |
| with/without | | | | |

A negative control with only gold particles yielded no spots.

### Example 4

### Transient Expression in Barley

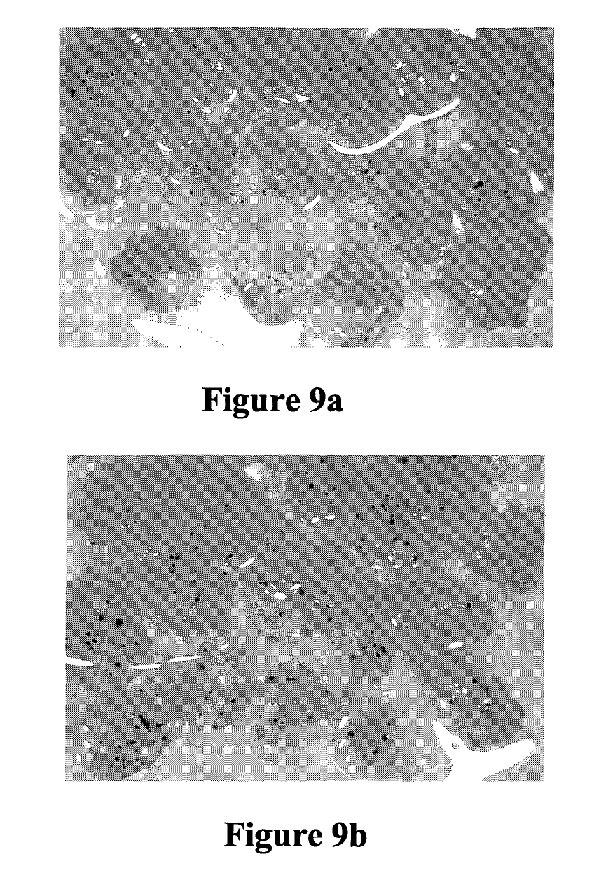

Other bombardment experiments were performed on barley embryos. The results also demonstrated that the presence of the FAD2 intron increases expression of the GUS protein in this species (FIG. 9). 2 plates of 16 embryos were bombarded with either pWP480 or pWP500 plasmid. The number of blue foci was scored per embryo and averaged.

Expression of co-bombarded GFP was again used to assess the relative efficiency of each individual bombardment and used to normalize and allow comparison between individual bombardments.

| | pWP480 | pWP500 |
|---|---|---|
| GFP | 63.9 | 60.7 |
| GUS | 27.00 | 92.2 |
| Ratio GUS/GFP | 0.42 | 1.52 |

| | |
|---|---|
| Ratio +/− intron | 3.61 |

### Example 5

### Stable Expression in Wheat

Wheat embryos were stably transformed by bombardment (WO 98/49316).

The embryos were bombarded with pWP461 or pWP464.

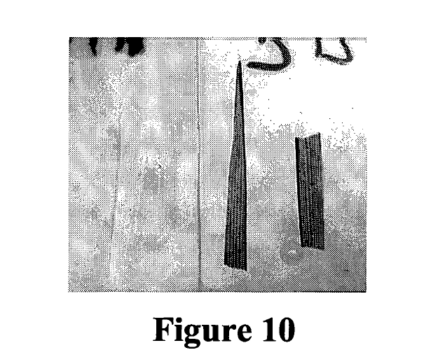

Transgenic plants were regenerated and grown, and expression of the GUS protein was studied by X-Gluc staining for glucuronidase activity. Following bombardment with WP461, no GUS expression was observed in regenerated transgenic plants. Following bombardment with WP464, strong constitutive expression could be observed in a range of tissues including leaf (FIG. 10), root, male and female floral parts and seed.

### Example 6

### Stable Transformation of Wheat

A selectable marker gene was prepared consisting of the Sc4 promoter, FAD 2 intron, the nptII coding region and nopaline synthase terminator. A comparable selectable marker gene was prepared consisting of the more widely used rice actin promoter and the associated first intron, the nptII coding region and nopaline synthase terminator. Both versions of the selectable marker gene were cloned into a suitable vector for wheat transformation. Wheat embryos were stably transformed by the Agrobacterium Seed Inoculation Method (SIM; WO 00/63398) using either

| Rice actin | Number of | Transgenic | |
|---|---|---|---|
| promoter + intron | embryos | events | Efficiency (%) |
| Experiment 6 | 192 | 1 | 0.5 |
| Experiment 7 | 206 | 19 | 9.2 |
| Experiment 8 | 220 | 3 | 1.4 |
| Experiment 9 | 225 | 4 | 1.8 |
| Experiment 10 | 145 | 1 | 0.7 |
| Experiment 11 | 139 | 18 | 13 |
| Total | 1127 | 46 | 4.1 |

version of the selectable marker, and stably transformed transgenic plantlets selected on plant tissue culture media containing a suitable concentration of Geneticin G418. The number of independent transgenic events was recorded and the transformation efficiency calculated as the percentage of treated embryos regenerating transgenic events.

| Sc4 promoter + | Number of | Transgenic | |
|---|---|---|---|
| FAD 2 intron | embryos | events | Efficiency (%) |
| Experiment 1 | 265 | 11 | 4.2 |
| Experiment 2 | 230 | 1 | 0.4 |
| Experiment 3 | 270 | 8 | 3.0 |
| Experiment 4 | 224 | 8 | 3.6 |
| Experiment 5 | 230 | 19 | 8.3 |
| Total | 1219 | 47 | 3.9 |

The rice actin promoter including the first intron is a very strong promoter widely used to drive the selectable marker in monocot species. Transformation frequencies were observed between 0.5 and 9.2% with an average of 4.1%. When the selectable marker was driven by the Sc4 promoter in combination with the FAD 2 intron, transformation frequencies of 0.4 to 8.3%, with an average of 3.9% were observed.

Surprisingly the combination of the very weak Sc4 promoter with the FAD2 intron is as efficient as the widely used rice actin promoter when used to drive the selectable marker needed for stable wheat transformation.

### Example 7

### Stable Transformation of Maize

Both versions of the selectable marker gene described in Example 6 were cloned into a suitable vector for maize transformation. Maize embryos were stably transformed essentially by the method of Ishida et al (1996) using either version of the selectable marker, and stably transformed transgenic plantlets selected on plant tissue culture media containing a suitable concentration of kanamycin. The number of independent transgenic events was recorded and the transformation efficiency calculated as the percentage of treated embryos regenerating transgenic events.

| | | Number of | Transgenic | |
|---|---|---|---|---|
| | | embryos | events | Efficiency % |
| Sc4 promoter + | | | | |
| FAD 2 intron | | | | |
| Experiment | 1 | 2656 | 62 | 2.33 |
| | 2 | 1455 | 12 | 0.82 |
| | 3 | 1196 | 21 | 1.76 |
| | 4 | 2212 | 30 | 1.36 |
| | 5 | 1306 | 8 | 0.61 |
| | 6 | 1363 | 24 | 1.76 |
| | 7 | 1247 | 24 | 1.92 |
| | 8 | 1307 | 17 | 1.30 |
| | 9 | 2297 | 18 | 0.78 |
| | 10 | 2177 | 20 | 0.92 |
| | 11 | 4306 | 67 | 1.56 |
| | 12 | 2016 | 23 | 1.14 |
| | 13 | 1218 | 6 | 0.49 |
| | 14 | 1529 | 9 | 0.59 |
| Total | | 26285 | 341 | 1.30 |
| Rice actin | | | | |
| promoter + | | | | |
| intron | | | | |
| Experiment | 1 | 1177 | 4 | 0.34 |
| | 2 | 870 | 6 | 0.69 |
| | 3 | 900 | 3 | 0.33 |
| | 4 | 685 | 4 | 0.58 |
| | 5 | 730 | 1 | 0.14 |
| | 6 | 698 | 1 | 0.14 |
| | 7 | 2349 | 7 | 0.30 |
| | 8 | 1895 | 23 | 1.21 |
| | 9 | 2515 | 42 | 1.67 |
| | 10 | 1863 | 17 | 0.91 |
| | 11 | 1448 | 10 | 0.69 |
| | 12 | 2231 | 9 | 0.40 |
| | 13 | 2170 | 20 | 0.92 |
| | 14 | 3067 | 30 | 0.98 |
| | 15 | 1135 | 4 | 0.35 |
| | 16 | 2287 | 5 | 0.22 |
| | 17 | 4132 | 54 | 1.31 |
| | 18 | 1218 | 6 | 0.49 |
| | 19 | 3250 | 12 | 0.37 |
| | 20 | 2024 | 20 | 0.99 |
| | 21 | 2013 | 20 | 0.99 |

| | | | |
|---|---|---|---|
| Total | 38657 | 298 | 0.77 |

Surprisingly the combination of the very weak Sc4 promoter with the FAD 2 intron is more efficient than the widely used rice actin promoter when used to drive the selectable marker needed for stable maize transformation (average 1.3% compared with 0.77%).

1. A genetic construct for the expression of a polypeptide in a plant cell, comprising:
(a) a promoter operative within said plant cell,
(b) the first intron of the FAD2 gene,
(c) a nucleotide sequence operably linked to said promoter, wherein said nucleotide sequence encodes said polypeptide,
(d) a polyadenylation site (PAA) operably linked to said nucleotide sequence.

2. The genetic construct of claim 1, wherein said FAD2 gene is from Arabidopsis thaliana (GenBank AJ271841), wherein said first intron is represented by SEQ ID No 1.

3. The genetic construct of claim 1, wherein said promoter is a tissue-specific promoter.

4. The genetic construct of claim 1, wherein said promoter is a constitutive promoter.

5. The genetic construct of claim 1, capable of integration within the genome of said plant cell.

6. A method for enhancing the expression of a polypeptide in a plant cell, comprising the step of introducing the genetic construct of claim 1 within said plant cell.

7. A method for increasing, in a plant cell, the expression rate of a gene under the control of a promoter, said method comprising the steps of:
(a) inserting the first intron of the FAD 2 gene between said promoter and the translation start of said gene, thereby forming an intron-modified gene; and
(b) introducing said intron-modified gene into a plant cell such that said gene is expressed under the control of said intron in said plant cell, whereby the expression rate of said gene is increased as compared to an unmodified gene.

8. The method of claim 6 wherein said intron has a nucleotide sequence as set forth in SEQ ID No 1 or functionally equivalent duplications or modifications in length or minor sequence variations that do not significantly effect function.

9. The use of the first intron of the FAD2 gene for enhancing expression of a polypeptide in a plant cell.


SEQUENCE LISTING (12 sequences)

SEQ ID NO: 1 - DNA, 1135 bp, Arabidopsis thaliana
gtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgatcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcag

SEQ ID NO: 2 - DNA, 3391 bp, Artificial
  proVir35Sx2 - EcGUS - terVirCaMV
ctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggctgcaggtcgacggatccccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccnntctagaattcggctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 3 - DNA, 4534 bp, artificial
  proVir35Sx2 - intAtFAD2 - EcGUS - terVirCaMV
ctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttggctgcaggtcgacggatccccgggtttgtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcagtggcccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccnntctagaattcggctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 4 - DNA, 3361 bp, artificial
  proVirSc4 - EcGUSintStLS1 - terVirCaMV
taattgttattatcaataaaagaatttttattgttattgtgttatttggtaatttatgcttataagtaattctatgattaattgtgaattaataagactaatgaggataataattgaatttgattaaattaactctgcgaagccatatgtctttcacgtgagagtcacgtgatgtctccgcgacaggctggcacggggcttagtattacccccgtgccgggatcagagacatttgactaaatgttgacttggaataatagcccttggattagatgacacgtggacgctcaggatctgtgatgctagtgaagcgcttaagctgaacgaatctgacggaagagcggacaaacgcacatggactatggcccactgctttattaaagaagtgaatgacagctgtctttgcttcaagacgaagtaaagaatagtggaaaacgcgtaaagaataagcgtactcagtacgcttcgtggctttataaatagtgcttcgtcttattcttcgttgtatcatcaacgaagaagttaagctgatcccgggaattcgcggcccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtcggatcccccgggctgcaggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 5 - DNA, 4561 bp, artificial
  proVirSc4 - intAtFAD2 - EcGUSintStLS1 - terVirCaMV
taattgttattatcaataaaagaatttttattgttattgtgttatttggtaatttatgcttataagtaattctatgattaattgtgaattaataagactaatgaggataataattgaatttgattaaattaactctgcgaagccatatgtctttcacgtgagagtcacgtgatgtctccgcgacaggctggcacggggcttagtattacccccgtgccgggatcagagacatttgactaaatgttgacttggaataatagcccttggattagatgacacgtggacgctcaggatctgtgatgctagtgaagcgcttaagctgaacgaatctgacggaagagcggacaaacgcacatggactatggcccactgctttattaaagaagtgaatgacagctgtctttgcttcaagacgaagtaaagaatagtggaaaacgcgtaaagaataagcgtactcagtacgcttcgtggctttataaatagtgcttcgtcttattcttcgttgtatcatcaacgaagaagttaagctgatcccgggaattcgcggccgctctagaactagtggatcccccgggccaggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgatcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcagaaacccgggctgcaggaattcgatatcaagcttcccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtcggatcccccgggctgcaggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 6 - DNA, 3307 bp, artificial
  proVirBSV - EcGUSintStLS1 - terVirCaMV
gatgctctagatctgctggatatcagtaatgatgactgaagcggaagtggcggacccctaccacgtgttgataccaaccggtgtgaagactgataagatgcggagtgagctggataccactcactttatgtaaagaggagacaaagtataatgtctctttattttaagtttgtcggtgtgcgttgtctagtcacgcacgatgacctttagtgactttgcaggattcttacgcaaagttgttaggccagagacatgtgatgatgcttatctgcattattggtggatgccacctaacgatgccagaaagctccacaactctctatataaggagccttgtattcaggttgcaaacacgcaccacaacgcgagtttactcctgatttgagaaataaaaacttctgtgcttgaaacacactttgtgcgagttcactttgtgcgagtagagcgcaagatcctagttccgcgagcgtagacccgtctggtatcagagcaagccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtcggatcccccgggctgcaggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 7 - DNA, 4259 bp, artificial
  proVirBSV - intAtFAD2 - EcGUS - terVirCaMV
tctagatctgctggatatcagtaatgatgactgaagcggaagtggcggacccctaccacgtgttgataccaaccggtgtgaagactgataagatgcggagtgagctggataccactcactttatgtaaagaggagacaaagtataatgtctctttattttaagtttgtcggtgtgcgttgtctagtcacgcacgatgacctttagtgactttgcaggattcttacgcaaagttgttaggccagagacatgtgatgatgcttatctgcattattggtggatgccacctaacgatgccagaaagctccacaactctctatataaggagccttgtattcaggttgcaaacacgcaccacaacgcgagtttactcctgatttgagaaataaaaacttctgtgcttgaaacacactttgtgcgagttcactttgtgcgagtagagcgcaagatcctagttccgcgagcgtagacccgtctgtcgacggatccccgggccaggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcagaaacccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccnntctagaattcggctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 8 - DNA, 4448 bp, artificial
  proTaHMWG - intAtFAD2 - EcGUSintStLS1 - terVirCaMV
agctttgagtggccgtagatttgcaaaagcaatggctaacagacacatattctgccaaaccccaagaaggataatcacttttcttagataaaaaagaacagaccaatatacaaacatccacacttctgcaaacaatacatcagaactaggattacgccgattacgtggctttagcagactgtccaaaaatctgttttgcaaagctccaattgctccttgcttatccagcttcttttgtgttggcaaactgcgcttttccaaccgattttgttcttctcgcgctttcttcttaggctaaacaaacctcaccgtgcacgcagccatggtcctgaaccttcacctcgtccctataaaagcctagccaaccttcacaatcttatcatcacccacaacaccgagcaccacaaactagagatccgaattaattctcgcggccgctctagaactagtggatcccccgggccaggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgatcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcagaaacccgggctgcaggaattcgatatcaagcttcccatggtacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcggtgacgtcggatcccccgggctgcaggaattcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggtgacggggat

SEQ ID NO: 9 - DNA, 25 bp, Artificial
  forward primer
gggccaggtccgtcgcttctcttcc

SEQ ID NO: 10 - DNA, 24 bp, artificial
  reverse primer
gggtttctgcagaaaaccaaaagc

SEQ ID NO: 11 - DNA, 19 bp, artificial
  forward primer
gatgctctagatctgctgg

SEQ ID NO: 12 - DNA, 29 bp, artificial
  reverse primer
ggccatggcttgctctgataccagacggg